(19) United States
(12) Patent Application Publication
(10) Pub. No.: US 2026/0053896 A1
(43) Pub. Date: Feb. 26, 2026

(54) WOUND HEALING AND RELATED PRODUCTS AND METHODS THEREOF FOR REGULATING TISSUE GROWTH FACTORS FOR HEALING

(71) Applicant: LifeSciencePLUS, Inc., Mountain View, CA (US)

(72) Inventors: Guiting Lin, San Francisco, CA (US); Stephen Grubb, Atherton, CA (US); Vicky Feng, Atherton, CA (US)

(21) Appl. No.: 19/104,220

(22) PCT Filed: Aug. 25, 2023
(86) PCT No.: PCT/US2023/031126

§ 371 Date: Feb. 16, 2025

Related U.S. Application Data
(60) Provisional application No. 63/402,296, filed on Aug. 30, 2022.

(51) Int. Cl. A61K 38/36; A61K 31/717; A61P 17/02

(52) U.S. Cl. CPC A61K 38/36; A61K 31/717; A61P 17/02

(57) ABSTRACT

The invention provides novel compositions and methods for tissue growth factor regulation for healing and tissue regeneration, and methods for their preparation and use.

### PRIORITY CLAIMS AND RELATED PATENT APPLICATIONS

This application claims the benefit of priority to U.S. Provisional Application Ser. No. 63/402,296, filed on Aug. 30, 2022, the entire content of which is incorporated herein by reference in its entirety.

### TECHNICAL FIELDS OF THE INVENTION

The invention generally relates to hemostatic agents, wound healing, bacteria static agents and tissue regeneration. More particularly, the invention relates to novel compositions and methods for tissue growth factor regulation for healing and tissue regeneration, and methods for their preparation and use thereof.

### BACKGROUND OF THE INVENTION

Wound healing, infection control and tissue repair are significant issues in health care. Tissue regeneration in humans is extremely limited and constitutes a major challenge to the proper repair of damaged tissue at a wound site. Chronic, non-healing wounds can cause prolonged morbidity. For example, tissue repair is particularly relevant to bedridden or diabetic patients who develop severe non-healing ulcers or patient with gum ulcers or stomach ulcers. Patients suffering from internal lesions, such as those associated with the digestive tract, are particularly susceptible to the effects of non or slow-healing tissue damage.

An ongoing need remains for novel and improved materials and devices that promote cell proliferation and tissue regeneration.

### SUMMARY OF THE INVENTION

This invention is based on the discovery of a revolutionary application of carboxymethylcellulose that is efficiently used in wound care for tissue regeneration. This invention can be widely used for full thickness burn wounds, surgical, trauma, diabetic ulcers wounds, acute and chronical wounds, and lesions of the foot and deep dermal filler or skin surface tissue regeneration. This synthetic polymer has been shown to be suitable for hemostatic application, wound sealing, and protection for skin wounds, such as burns.

In one aspect, the invention generally relates to a composition for use in modulating one or more growth factors for skin and/or tissue regeneration, comprising a biocompatible etherified sodium carboxymethyl cellulose (CMC).

In yet another aspect, the invention generally relates to a kit for skin or tissue injury or burn treatment, comprising a medical device of the invention.

In yet another aspect, the invention generally relates to a method for modulating one or more growth factors for skin and/or tissue regeneration, comprising applying a composition or a medical device of the invention to a patient in need thereof at an injury or burn site.

In yet another aspect, the invention generally relates to a method for modulating one or more growth factors for promoting dermal and/or stem cell proliferation, comprising applying a composition or a medical device of any of the invention to a patient in need thereof at an injury or burn site.

BloodSTOP iX Wound Heal (BSiX) is demonstrated herein to be an ideal solution for wound hemostasis and healing, offering a multifaceted approach to promote tissue regeneration. By inducing a range of endogenous cytokines, with particular emphasis on VEGF and PDGF, BSiX plays a vital role in accelerating tissue healing. Simultaneously, it effectively inhibits inflammatory cytokines like CINC-1, CINC-2, and MMP-8, thereby reducing the risk of wound infection and facilitating the healing process. We have performed studies to quantify the changes in these cytokines with the application of BSiX.

Modulating endogenous cytokines: The BloodSTOP iX Wound Heal gel provide perfect microenvironment for BloodSTOP iX Wound Heal molecules to bind to cellular receptors on cell membranes to activate cellular signaling for modulating 43 cytokines, including VEGF, PDGF, MMP8, TGFb3 et al.

Scaffolding effect: When the BloodSTOP iX Wound Heal meet the blood, it immediate trig the hemostatic process and turn into gel to seal the site of bleeding. The BloodSTOP iX Wound Heal gel will further ensure the hemostasis. Presented as gel, the BloodSTOP iX Wound Heal serves as a scaffold, giving the patient's native cells a place to live, thrive and rebuild tissue. Without cross-linking, the open scaffold allows for cellular infiltration and capillary growth.

Promoting quality angiogenesis and well healing: VEGF and PDGF, the cytokines stimulated by BloodSTOP iX Wound Heal, were released from the host cells and rebuilds well-vascularized new tissues.

Providing the foundation for skin grafting and re-epithelialization: TGFβ3 and others, the cytokine stimulated by BloodSTOP iX Wound Heal, facilitate cellular transformation and infiltration to enhance skin regeneration and re-epithelialization.

Protecting tissue cells from apoptosis and damages from injury: FGF7 and others, the cytokine stimulated by BloodSTOP iX Wound Heal, protect tissue from injury and decrease apoptosis.

Helping reduce infection risk: BloodSTOP iX Wound Heal decrease inflammatory cytokines, including MMP8, CINC1 and CINC3, to reduce the infection and inflammation risk.

### BRIEF DESCRIPTION OF THE DRAWINGS

FIG. 1. BSiX significantly improved skin healing ability after burn injury. Upon closer examination, high-magnification images revealed the presence of nascent epithelial cells that overlaid the granulation tissue, effectively filling the gap between the skin and underlying tissue.

FIG. 2. BSiX significantly improved skin healing ability after burn under high magnification, new epithelial cells can be seen covering the granulation tissue, filling the space between the skin and the tissue.

FIG. 3. BSiX promoted the formation of many new blood vessels in the subcutaneous tissue.

FIG. 4. BSiX increased the expression of endogenous vascular endothelial growth factor (VEGF) in subcutaneous tissue. (a). Immunohistochemistry (IHC) analysis of Vascular Endothelial Growth Factor (VEGF) was performed in the Normal (N), Vaseline, and BSiX groups. Positive expression of VEGF is indicated by the presence of a brown signal. (b) The expression of VEGF is depicted in a histogram, representing the distribution of VEGF-positive expression signals in each group (n-6). The Y-axis represents the percentage (%) of VEGF-positive expression signal value relative to the total field of view. (*P<0.05).

FIG. 5. BSiX increased the expression of endogenous Platelet-Derived Growth Factor (PDGF) in subcutaneous tissue. (a) Immunohistochemistry (IHC) analysis was conducted to examine the expression of Platelet-Derived Growth Factor (PDGF) in the Normal (N), Vaseline, and BSiX groups. The positively expressed PDGF is visualized by a brown signal. (b) The expression of PDGF is depicted in a histogram, presenting the distribution of PDGF-positive expression signals in each group (n=6). The Y-axis represents the percentage (%) of the PDGF-positive expression signal value relative to the total field of view. (*P<0.05).

FIG. 6. BSiX increased the expression of endogenous Epidermal Growth Factor (EGF) in subcutaneous tissue. (a) Immunohistochemistry (IHC) analysis revealed the expression of Epidermal Growth Factor (EGF) in the Normal (N), Vaseline, and BSiX groups. The positively expressed EGF is indicated by a brown signal. (b) The expression of EGF is depicted in a histogram, illustrating the distribution of EGF-positive expression signals in each group (n=6). The Y-axis represents the percentage (%) of the EGF-positive expression signal value relative to the total field of view.

FIG. 7. BSiX increased the expression of endogenous basic fibroblast growth factor (FGF2) in subcutaneous tissue. (a). IHC result of FGF2 in N, Vaseline and BSiX group. Brown signal is positively expressed FGF2. (b) Expression of FGF2 presented in a histogram (n=6, in each group) Y axis: the percentage (%) of the value of FGF2 positive expression signal in the value of the total field of view. (*P<0.05).

FIG. 8. BSiX increased the expression of endogenous Keratinocyte Growth Factor (FGF7) in subcutaneous tissue. (a). IHC result of FGF7 in N, Vaseline and BSiX group. Brown signal is positively expressed FGF7. (b) Expression of FGF7 presented in a histogram (n=6, in each group) Y axis: the percentage (%) of the value of FGF7 positive expression signal in the value of the total field of view. (*P<0.05).

FIG. 9. BSiX increased the expression of endogenous Transforming Growth Factor β1 (TGF-β1) in subcutaneous tissue. (a). IHC result of TGF-β1 in N, Vaseline and BSiX group. Brown signal is positively expressed TGF-β1. (b) Expression of TGF-β1 presented in a histogram (n=6, in each group) Y axis: the percentage (%) of the value of TGF-β1 positive expression signal in the value of the total field of view.

FIG. 10. BSiX increased the expression of endogenous Transforming Growth Factor β3 (TGF-β3) in subcutaneous tissue. (a). IHC result of TGF-β3 in N, Vaseline and BSiX group. Brown signal is positively expressed TGF-β3. (b) Expression of TGF-β3 presented in a histogram (n=6, in each group) Y axis: the percentage (%) of the value of TGF-β3 positive expression signal in the value of the total field of view. (*P<0.05).

FIG. 11. Cytokine microarray scanned Images.

FIG. 12. BSiX regulated tissue endogenous cytokines and promotes tissue regeneration in burn injury—3 days.

FIG. 13. BSiX regulated tissue endogenous cytokines to promote tissue regeneration after burn—3 days. Comparison with Vaseline Gauze.

FIG. 14. BSiX regulated tissue endogenous cytokine (classification)—3 days.

FIG. 15. BSiX regulated tissue endogenous cytokine-binding receptor (classification)—3 days.

FIG. 16. BSiX regulated tissue and extracellular matrix-associated endogenous cytokines—3 days.

FIG. 17. BSiX regulated tissue endogenous cytokine-related cell signaling pathways—3 days.

FIG. 18. BSiX regulated tissue endogenous cytokines and promotes tissue regeneration in burn injury—20 days.

FIG. 19. BSiX regulated tissue endogenous cytokines to promote tissue regeneration after burn—20 days. Comparison with Vaseline Gauze.

FIG. 20. BSiX regulated tissue endogenous cytokine (classification)—20 days.

FIG. 21. BSiX regulated tissue endogenous cytokine-binding receptor (classification)—20 days.

FIG. 22. BSiX regulated tissue and extracellular matrix-associated endogenous cytokines—20 days.

FIG. 23. BSiX regulated tissue endogenous cytokine-related cell signaling pathways—20 days.

FIG. 24. BSiX regulated tissue endogenous cytokines, inhibits inflammatory response and promotes tissue regeneration in the treatment of burn injury.

### DETAILED DESCRIPTION OF THE INVENTION

The invention is based in part on the unexpected discovery of BloodSTOP iX Wound Heal as an effective composition for modulating growth factors for skin and/or tissue regeneration.

Previous studies have shown the nanostructure of the BloodSTOP iX Wound Heal (BSiX) (LifeSciencePlus. Inc., CA, USA) nanocellulose matrix. Preclinical results showed that BSiX promoted burn wound healing in two animal models. In clinical trials, BSiX reduced the recurrence of bleeding and burning/pain sensation after application, while anti-infective effects were also noted. Even more strikingly, BSiX promotes skin epithelization and improves scar tissue. Very recently, it has been noted that this polymer promoted adipose derived stem cell proliferation and enhanced wound healing. More interestingly, it binds to epithelium and promotes cell migration for tissue regeneration. Over the years, CMC, has been used as a single material with or without combination with drugs and co-excipients in partial thickness wounds, diabetic ulcers, lesions of the foot and deep dermal filler. These effects were observed at both donor and acceptor sites. At the same time, in vitro studies have shown that previous studies have shown that BSiX promotes mitosis and proliferation of skin stem cells in a dose-dependent manner. Activation of the Wnt/β-catenin signaling pathway by AMPK. This may be a potential mechanism by which BSiX activates stem cells and dermal cells to promote wound healing.

Therefore, BSiX and related products have the demonstrated ability to stimulate dermal and stem cell proliferation and thus are useful for enhancing skin regeneration after skin injury or burn. Activation of residential stem/progenitor cells is a novel approach for stem cells therapy on the skin, however, there is no well-accepted method to achieve this task, especially to achieve this task by modulating tissue growth factors.

In one aspect, the invention generally relates to a composition for use in modulating one or more growth factors for skin and/or tissue regeneration, comprising a biocompatible etherified sodium carboxymethyl cellulose (CMC).

In certain embodiments, the skin or tissue comprises an injury or burn.

In certain embodiments, tissue regeneration is promoted by dermal and/or stem cell proliferation.

In certain embodiments, the CMC is characterized by a degree of fabric substitution in the range about 0.1 to about 0.99 (e.g., about 0.1 to about 0.8, about 0.1 to about 0.5, about 0.2 to about 0.99, about 0.3 to about 0.99, about 0.2 to about 0.9, about 0.2 to about 0.8).

In certain embodiments, the CMC is characterized by a degree of polymerization is from about 50 to about 2,000 (from about 50 to about 1,000, from about from about 50 to about 500, from about 100 to about 2,000, from about 500 to about 2,000, from about 100 to about 1,500, from about 100 to about 800).

In certain embodiments, the CMC is characterized by a sodium content in the range from about 6.5% to about 9.5% (e.g., about 6.5%, 7.0%, 7.5%, 8.0%, 8.5%, 9.0%, 9.5%).

In certain embodiments, the composition of the invention is suitable for use in stimulating endogenous cytokines VEGF and/or PDGF.

In certain embodiments, the composition of the invention is suitable for use in inhibiting one or more inflammatory cytokines (e.g., MMP8, CINC1 and CINC3).

In another aspect, the invention generally relates to a medical device comprising the composition of the invention.

In certain embodiments, the medical device further comprises one or more bioactive agent(s) (e.g., VEGF, PDGF and EGF) that stimulates wound healing.

In certain embodiments, the medical device further comprises one or more bioactive agent(s) (e.g., MMP8, CINC1 and CINC3) that prevents or reduces infection.

In certain embodiments, the medical device further comprises one or more anti-fibrinolysis agents (e.g., FGF7 and FGF2).

In certain embodiments, the medical device further comprises one or more adhesive agents (e.g., PDGF, VEGF).

In certain embodiments, the medical device further comprises one or more coagulation factors (e.g., VIII and XIII).

In certain embodiments, the medical device is in a form selected from powders, fibers, webs, nonwoven cloths, sponges, films, capsules, pellets, columns, plugs and colloids.

In yet another aspect, the invention generally relates to a kit for skin or tissue injury or burn treatment, comprising a medical device of the invention.

In yet another aspect, the invention generally relates to a method for modulating one or more growth factors for skin and/or tissue regeneration, comprising applying a composition or a medical device of the invention to a patient in need thereof at an injury or burn site.

In yet another aspect, the invention generally relates to a method for modulating one or more growth factors for promoting dermal and/or stem cell proliferation, comprising applying a composition or a medical device of any of the invention to a patient in need thereof at an injury or burn site.

In certain embodiments of methods of the invention, the composition or medical device is applied on the patient in need thereof for about 1 days to about 28 days (e.g., about 1 to 7 days, about 7 to 14 days, about 14 to 21 days, about 21 to 28 days).

### EXAMPLES

The Examples below describe certain exemplary embodiments of compounds prepared according to the disclosed invention. It will be appreciated that the following general methods, and other methods known to one of ordinary skill in the art, can be applied to compounds and subclasses and species thereof, as disclosed herein.

### Materials and Methods

### BloodSTOP iX Wound Heal

The terms “BloodSTOP iX Wound Heal” and “BSiX”, as used herein, refer to a biocompatible, water-soluble matrix composition or material comprising etherified sodium carboxymethyl cellulose.

BSiX has several new isoforms, including:
1) Thickness, BSiX is ranged from about 0.1 mm to about 2 mm;
2) Texture, including non-woven, plain weave, twill weave;
3) Size, ranged from about 0.1 to about 10 inch; and
4) Degree of substitution, ranged from about 0.1 to about 0.99.

### Establishment of Animal Model

A total 48 SD rats weighing 190±10 g were randomly divided into 8 groups including sham group, control groups and experimental groups (time points 3, 14 and 20 days).
1) Sham surgical group (N)
2) Control Vaseline gauze—1 (Vaseline): 3 days
3) Control Vaseline gauze—2 (Vaseline): 14 days
4) Control Vaseline gauze—3 (Vaseline): 20 days
5) Control Regular gauze (Gauze control): 20 days
6) BloodSTOP iX Wound Heal—1 (BSiX): 3 days
7) BloodSTOP iX Wound Heal—2 (BSiX): 14 days
8) BloodSTOP iX Wound Heal—3 (BSiX): 20 days

The rats were prohibited from water ingestion for 12 hours before burns injury. The back of each rat was shaved and induced burn injury in the flask with water at 100° C. (diameter 2 cm, thickness 0.2 mm) for 8 seconds. After modeling, in the control Vaseline gauze group, the injured site was covered with 2 layers of Vaseline gauze; in the control regular gauze group, the injured site was covered with 2 layers of regular gauze, and the experimental group were covered with 2 layers of BSiX. All wounds were wrapped with gauze and fixed. Daily observation and images were taken to observe the injured site.

Starting from the second day, the dressing was changed every day. The pictures were taken every day to observe and record the process of wound heal. The skin specimens of the control and treatment group were obtained on the 3, 14, and 20 days after the treatment in the injury model.

### Tissue Sample Collection

For the rats to be collected tissue samples, animals were anesthetized with 10% chloral hydrate by intraperitoneal injection, and then tissue scissors were used to cut tissues along 5 mm out of the edge of the burn wound. The skin tissue, including the thickness of the whole skin and the deep muscle part, were collected and placed in Bouin's Fixation with liquid or Zenker's solution for histological study and in PBS for Cytokine Array study.

### HE Staining

The integrity of the skin structure, the number of neutrophils, fibroblasts and capillaries, and the repair of the dermis, epidermis and skin appendages were checked. Also, the presence of bacteria in the tissue were also checked.

To prepare the sections for analysis, they were initially stained with Hematoxylin dye solution for 5 minutes and then rinsed with clean water for 5 minutes. Subsequently, the sections were stained with a 1% hydrochloric acid alcohol color separation solution for 7-9 seconds, followed by a thorough wash with clean water for 10 minutes. The slides were then placed in double distilled water for 5 minutes. The process of dehydration involved sequentially immersing the slides in 70% alcohol, 80% alcohol, and 95% alcohol I, each for 3 minutes. Next, the slides were exposed to Eosin for staining, followed by immersion in 95% alcohol II and anhydrous alcohol I and II, each for 3 minutes. For transparency, the slides underwent a series of treatments: xylene, absolute alcohol (1:1) mixture, xylene, and 95% alcohol II, with each step lasting 3 minutes.

### Immunohistochemistry

The paraffin sections were dewaxed following the antigen retrieval. It was important to prevent excessive evaporation of the buffer solution and ensure that the slides did not dry out during this process. Once naturally cooled, the slides were immersed in PBS (pH 7.4), shaken, and washed three times for 5 minutes each. Subsequently, the endogenous peroxidase activity was blocked. The primary antibodies, including PDGF (dilution ratio 1:50), EGF (dilution ratio 1:200), VEGF (dilution ratio 1:200), FGF-2 (dilution ratio 1:200), FGF-7 (dilution ratio 1:400), TGF-β1 (dilution ratio 1:500), and TGF-β3 (dilution ratio 1:200), were then incubated with the slides overnight at 4° C. in a wet box. Following incubation, the slices were washed three times with PBS (pH 7.4) for 5 minutes each. The corresponding secondary antibodies (HRP-labeled) for each species of primary antibody were applied to the slides and incubated at room temperature for 15 minutes. Subsequently, the horseradish enzyme-labeled streptavidin working solution was added and incubated at room temperature for 15 minutes to initiate DAB color development. Positive staining was indicated by a brownish-yellow color, and the sections were rinsed with tap water to halt color development.

For image analysis, five randomly selected fields per tissue were captured and recorded using a Retiga Q Image digital still camera and ACT-1 software (Nikon Instruments Inc., Melville, NY). The differences in the expression of the seven growth factors among the different groups of skin tissues were then detected and analyzed.

### Cytokine Array

The total protein was isolated from tissues samples, each group of protein samples were pooled together, and cytokine array were used to detect cytokine changes between groups. The RayBio® C-Series Rat Cytokine Antibody Array C2 (RayBiotech Life, Inc. Peachtree Corners, GA. USA) was used for the semi-quantitative detection of 34 rat cytokines, including Activin A, Agrin, B7-2, CD86, b-NGF, CINC-1, CINC-2 alpha, CINC-3, CNTF, Fas Ligand, Fractalkine, GM-CSF, ICAM-1, IFN-gamma, IL-1 alpha, IL-1 beta, IL-1 R6, IL-2, IL-4, IL-6, IL-10, IL-13, Leptin, LIX, L-Selectin, MCP-1, MIP-3 alpha, MMP-8, PDGF-AA, Prolactin R, RAGE, Thymus, Chemokine-1, TIMP-1, TNF alpha, and VEGF-A. The membrane of cytokine array was incubated with 2 ml of blocking buffer at room temperature for 1 h. After removing the blocking solution with a suction pump, add 1000 μl sample (the sample is loaded at 500 μg/ml). Cover with a lid, wrap with plastic film, and incubate at 4 overnight with shaking. After washing, the cytokine array was incubated with biotin-labeled antibodies at room temperature for 2 hours. Then, 2 ml of HRP-streptavidin was added to each membrane and incubate with shaking at room temperature for 2 hours. The color was developed with solution C/D mixture. Image Quant LAS 4000 scanning chemiluminescence imaging analysis system (GE Healthcare Corporate, USA) was used to capture the images. The analysis software that comes with the instrument is used to extract data, and the data analysis software of AAR-CYT-2 was used for data pre-analysis.

For the differential gene expression screening, after the original data was normalized by software, select Normalization 2 without background data for analysis. Use fold change (fold change in expression) to screen differential proteins, and the selection conditions are as follows: 1) fold Change=<0.83 or fold Change>=1.2. 2) It was recommended to select an average (fluorescence) signal value >150 per group. The scatter plot function is ggplot2, and the data package is ggfortify, which comes from R software.

Gene Ontology (GO for short) is an international standard classification system for gene function. GO can be divided into three parts: molecular function, biological process and cellular component. Through GO enrichment analysis, it is possible to find important functions that lead to trait changes and find the genes corresponding to the functions. For the GO enrichment analysis of differential genes, the method adopted is fisher's exact test, and the data package is clusterProfiler, which comes from R/Bioconductor. The selection criteria are the number of proteins that fall on a certain term/GO difference >=2, p<0.05, The term/GO obtained in the drawing is sorted in descending order according to the value of Count, and the first 10 results are taken.

### Statistical Analysis

Data were analyzed with Prism 5 (GraphPad Software, San Diego, CA) and presented as means±standard deviation (S. D). Statistical significance between two groups was analyzed by Student t-test. For statistical significance among multiple groups, one-way ANOVA analysis followed by Bonferroni post hoc analysis was performed.

### RESULTS

### BSiX Promoted Tissue Healing and Angiogenesis

In this experiment, tissue paraffin sections were subjected to routine Hematoxylin and Eosin (HE) staining to evaluate tissue regeneration and vascular density in the subcutaneous tissue following a 20-day burn injury.

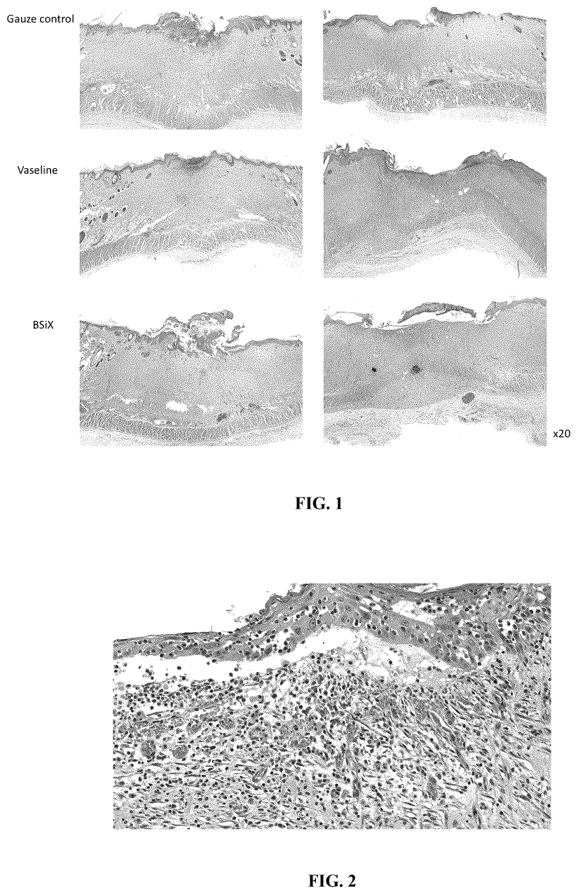

The results revealed that, compared to the Vaseline gauze and sham groups, the application of BSiX significantly enhanced the skin's healing capacity after burn injury. Notably, a newer epithelium with a crawling pattern was observed to migrate across the wound surface (FIG. 1). Upon closer examination at higher magnification, nascent epithelial cells were observed overlaying the granulation tissue, effectively filling the space between the skin and the underlying tissue. Furthermore, BSiX exhibited a remarkable ability to promote the formation of numerous new blood vessels within the subcutaneous tissue, facilitating tissue regeneration and recovery following the burn injury (FIG. 2).

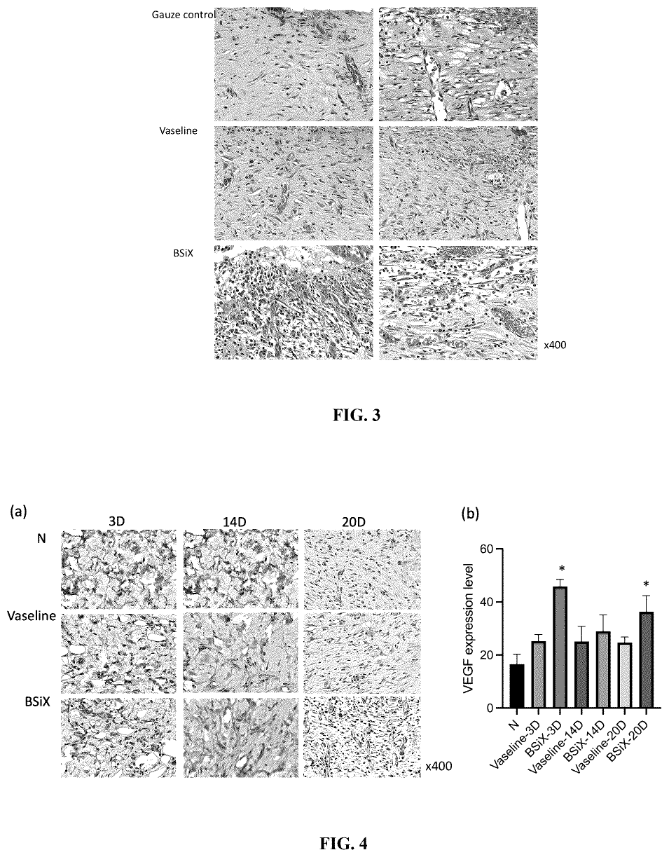

Most notably, BSiX promoted the formation of a large number of new blood vessels in the subcutaneous tissue and promoted tissue regeneration and recovery post the burn injury (FIG. 3).

### BloodSTOP iX Wound Heal Modulated Tissue Growth Factors

In this experiment, the effect of BSiX on the expression of 7 endogenous cytokines in tissue was determined by detecting in the tissue paraffin section. As previously described, 6 tissue specimens from each group were sectioned, followed by histoimmunochemical (IHC) staining. The stained tissue slides were subjected to holographic scanning, and the images were subjected to quantitative analysis of positive signals. At the same time, the effect of BSiX on tissue healing and regeneration in burn injury was also determined by detecting 43 endogenous cytokines in the tissue with Cytokine Array.

### Effects of BSiX on Vascular Epithelial Growth Factor (VEGF)

The Vascular epithelial growth factor (VEGF) is one of the dominated growth factors that the body stimulates and promotes angiogenesis.

The results of the study demonstrated that BSiX had a significant impact on the expression of VEGF in the tissue compared to the application of Vaseline gauze. Specifically, on the third day and 20^{th} days of application, BSiX markedly increased the expression of VEGF in the tissue (P<0.01) when compared to the Vaseline gauze group. Furthermore, on the 14^{th} day, BSiX continued to enhance the expression of VEGF in the tissue, although the differences were not statistically significant when compared to the Vaseline gauze group (P>0.05). However, when compared to the normal control group, BSiX significantly increased the expression of VEGF in the tissue at 3, 14, and 20 days (P<0.05) (FIG. 4). These findings highlight the ability of BSiX to positively influence the expression of VEGF, signifying its potential role in promoting angiogenesis and supporting tissue healing and regeneration.

### Effects of BSiX on Platelet-Derived Growth Factor (PDGF)

The Platelet-derived growth factor (PDGF) is one of growth factors that regulate cell growth and division. Specifically, PDGF plays an important role in angiogenesis, growth of blood vessels from pre-existing vascular tissue, mitosis, i.e., proliferation of mesenchymal cells, such as fibroblasts, osteoblasts, tenocytes, vascular smooth muscle cells, and the chemotaxis and directional migration of mesenchymal cells.

The findings of the study indicated that BSiX exhibited an increasing trend in the expression of platelet-derived growth factor (PDGF) in the tissues on the 3rd, 14th, and 20th days of application. Although the differences did not reach statistical significance when compared to the Vaseline gauze group (P>0.05), it is noteworthy that the effect was particularly prominent on the third day. In comparison to the normal control group, BSiX significantly enhanced the expression of PDGF in the tissues at 3, 14, and 20 days (P<0.05) (FIG. 5). These results suggest that BSiX has the potential to positively influence the expression of PDGF, indicating its potential role in promoting cell growth, angiogenesis, and the migration of mesenchymal cells.

### Effects of BSiX on Epidermal Growth Factor (EGF)

The Epidermal growth factor (EGF) is a cytokine that stimulates cell growth and differentiation by binding to its receptors and is closely related to tissue regeneration.

The study results revealed that on the 14th day of application, BSiX demonstrated an increase in the expression of epidermal growth factor (EGF) compared to Vaseline gauze.

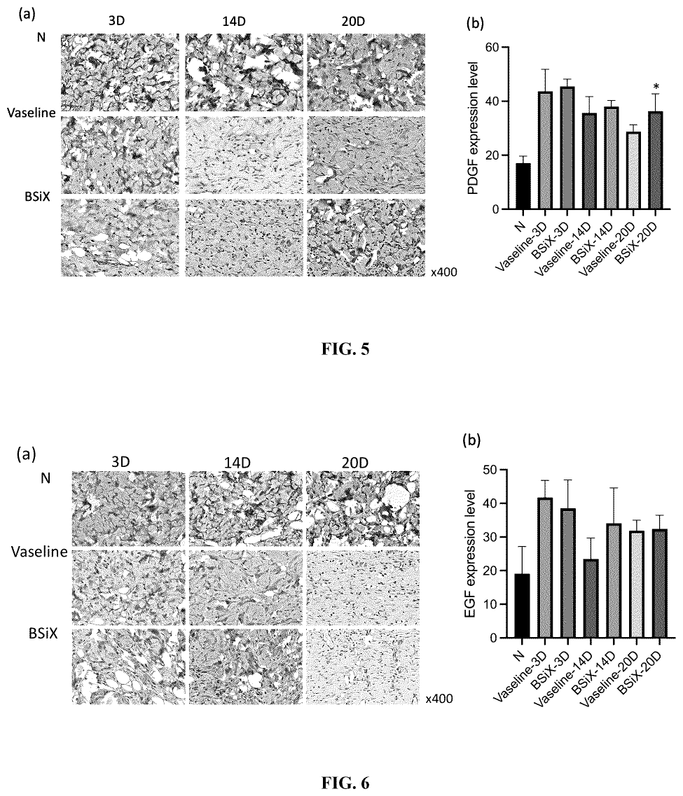

Although it was higher than the expression observed with Vaseline gauze, the difference did not reach statistical significance (P>0.05). On the 20th day, BSiX exhibited a slight elevation in the expression of EGF, slightly surpassing that of Vaseline gauze. It is important to note that on day 3, the effect of Vaseline gauze in promoting EGF expression appeared to be higher than that of BSiX, although the difference did not reach statistical significance (P>0.05). When compared to the normal control group, both BSiX and Vaseline gauze increased the expression of EGF in the tissues at 3, 14, and 20 days, but the differences did not reach statistical significance (P>0.05) (FIG. 6). These findings indicate that both BSiX and Vaseline gauze have the potential to influence the expression of EGF in tissue, suggesting their involvement in tissue regeneration processes. However, further investigation is required to ascertain the significance and clinical implications of these observations.

### The Effect of BSiX on Basic Fibroblast Growth Factor (FGF2)

The Basic fibroblast growth factor (FGF2) has broad mitogenic and cell survival activities and is involved in a variety of biological processes including embryonic development, cell growth, morphogenesis, tissue repair, tumor growth and invasion.

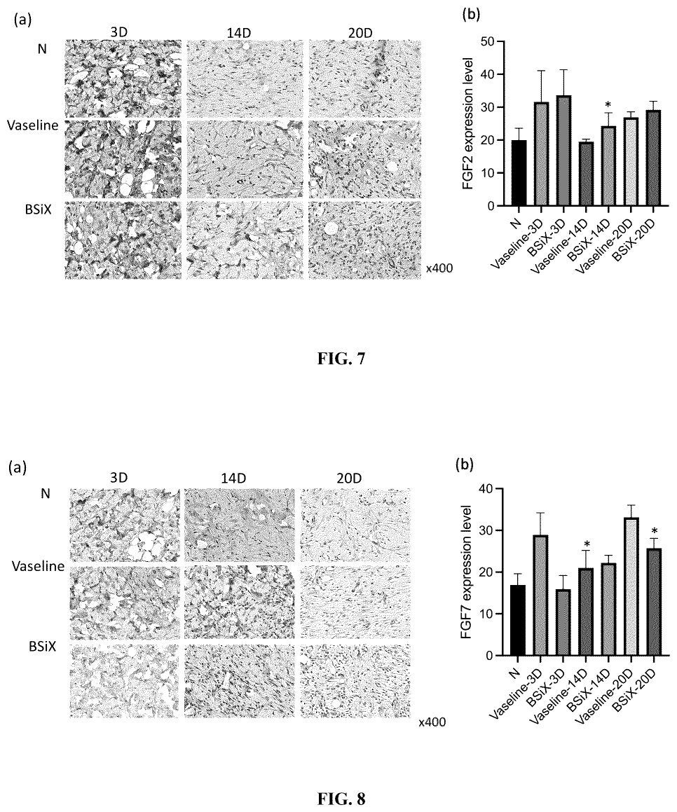

The study results indicate that BSiX led to an increase in the expression of basic fibroblast growth factor (FGF2) at 3, 14, and 20 days compared to the normal control group, but the differences did not reach statistical significance (P>0.05) except that at 14 days. Although the effect of BSiX was slightly higher than that of Vaseline gauze, no significant difference was observed (P>0.05) (FIG. 7). These findings suggest that BSiX may have a potential influence on the expression of FGF2, highlighting its possible role in promoting tissue repair and regeneration. However, further investigations are necessary to elucidate the clinical significance and underlying mechanisms associated with these observations.

### Effects of BSiX on Keratinocyte Growth Factor (FGF7)

The Keratinocyte growth factor (FGF7), also known as KGF (keratinocyte growth factor), is induced in mesenchymal cells by inflammation or tissue damage and is associated with tissue damage.

The results demonstrated that Vaseline gauze significantly enhanced the expression of keratinocyte growth factor (FGF7) on days 3 and 20 when compared to normal controls (P<0.05). On the other hand, BSiX did not exhibit any effect on keratinocyte growth factor (FGF7) expression on the third day and 20th day (P<0.05). However, BSiX showed a slight increase in the expression of keratinocyte growth factor (FGF7) on the 14th day. Nonetheless, these differences did not reach statistical significance when compared to normal tissue (P>0.05). These findings suggest that BSiX may possess a certain degree of protective function against tissue damage (FIG. 8). Further investigations are required to fully elucidate the potential role of BSiX in modulating keratinocyte growth factor (FGF7) expression and its implications for tissue healing and repair.

### Effects of BSiX on Transforming Growth Factor β1 (TGF-β1)

The Transforming Growth Factor β1 (TGF-β1) is a secreted protein with a variety of cellular functions, including the control of cell growth, cell proliferation, cell differentiation and apoptosis. It is closely related to tissue fibrosis and scarring.

The results revealed that Vaseline gauze significantly increased the expression of transforming growth factor β1 (TGF-1) on days 3 and 20 in comparison to normal controls.

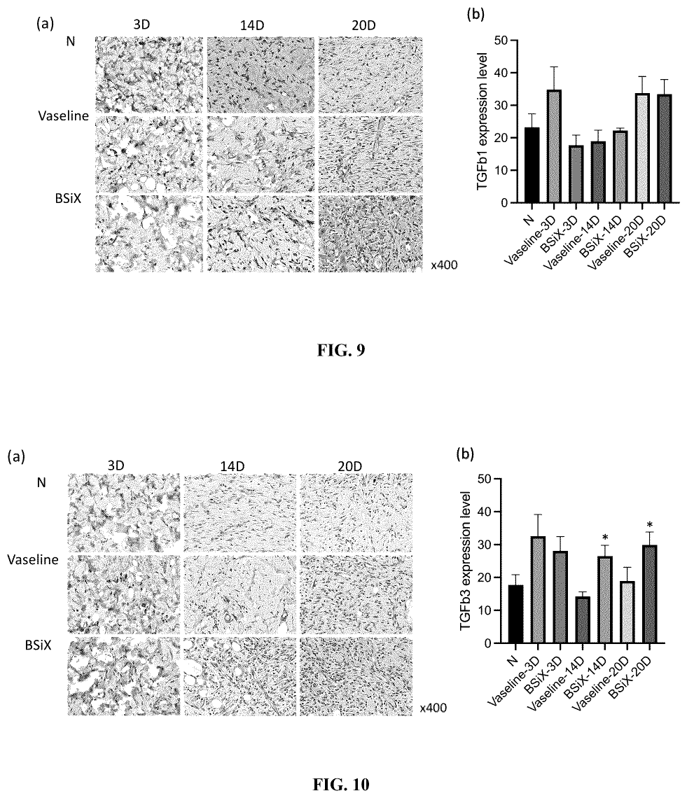

Interestingly, on the third day, BSiX exhibited a certain inhibitory effect on the expression of transforming growth factor β1 (TGF-β1). However, on the 14th and 20th days, BSiX slightly increased the expression of transforming growth factor β1 (TGF-1), although the comparison with normal tissues did not reach statistical significance (P>0.05). These findings suggest that BSiX may have a certain inhibitory effect on tissue fibrosis and scarring (FIG. 9). Further investigations are required to fully understand the potential impact of BSiX on modulating the expression of transforming growth factor β1 (TGF-β1) and its implications for fibrosis and scarring in tissue healing and regeneration.

### Effects of BSiX on Transforming Growth Factor β3 (TGF-β3)

The Transforming growth factor β3 (TGF-β3) is a pleiotropic cytokine that can regulate immune function, proliferation, especially epithelial-mesenchymal transition, and plays an important role in the repair of burn skin epithelium.

The results indicated that both BSiX and Vaseline gauze increased the expression of transforming growth factor β3 (TGF-β3) on day 3 compared to normal controls. However, on the 14th and 20th days, the effect of Vaseline gauze in increasing the expression of transforming growth factor β3 (TGF-β3) diminished, while BSiX continued to enhance the expression of transforming growth factor β3 (TGF-β3), showing a gradual increasing trend. Notably, on the 14th day and 20th, the effect of BSiX on the expression of transforming growth factor β3 (TGF-β3) was significantly higher than that of Vaseline gauze (P<0.05). These results suggest that BSiX exhibits a regulatory effect on the epithelial-mesenchymal transition of the skin epithelium, thereby promoting the formation and maturation of new skin (FIG. 10).

### Effects of BSiX on Multiple Tissue Growth Factors at Different Time Points

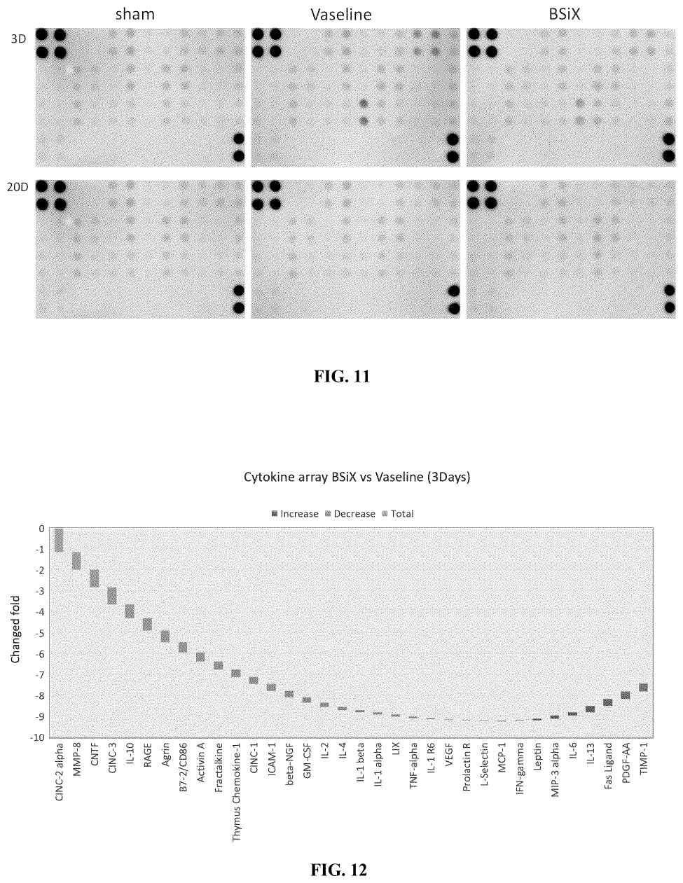

As mentioned above, tissue protein was extracted from 6 tissue samples in each group, and then each group was mixed together to form a test sample. The original scan of the detection results is shown in FIG. 11.

### Three-Day Comparison of BSiX and Vaseline Gauze Groups

Three days post the burn injury, the results showed that a total of 8 cytokines were increased by BSiX, including MCP-1, INF-r, Leptin, MIP3, IL6, IL 13, Fas-L, PDGF-AA, TIMP-1. The increase of PDGF-AA has important biological significance, indicating that BSiX can promote tissue regeneration.

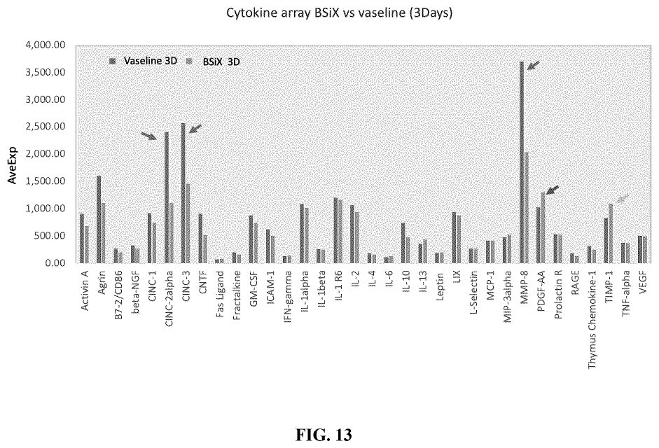

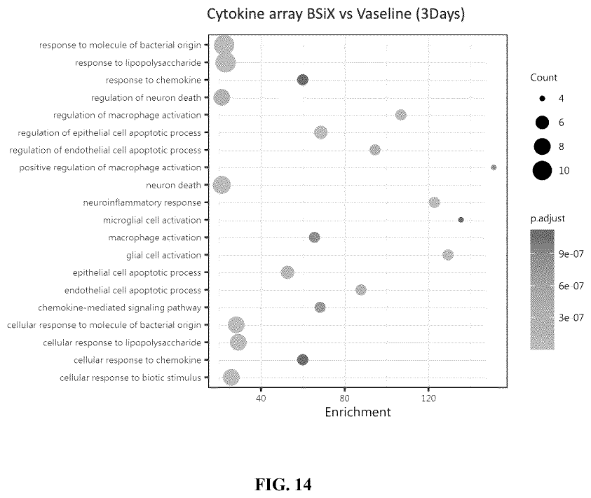

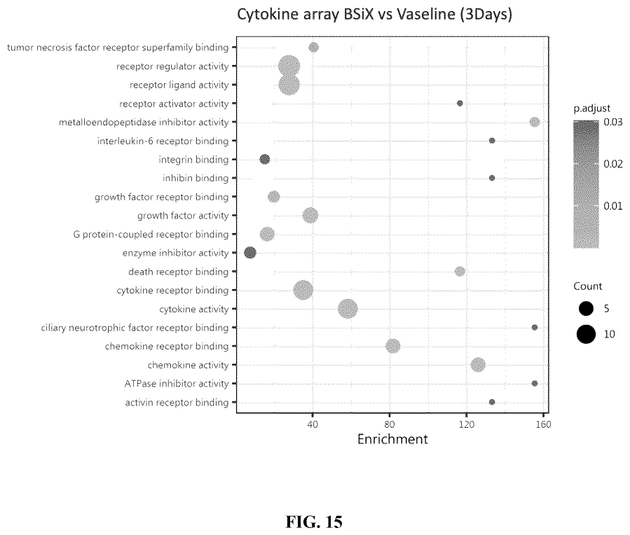

In addition, BSiX significantly inhibited 25 endogenous cytokines, the most important of which were CINC-2, MMP8, and CINC3, which were all related to tissue inflammation, indicating that BSiX could significantly inhibit tissue inflammation. (FIG. 12 & FIG. 13) Through the in-depth analysis of these endogenous cytokines, it was found that the factors related to bacterial infection and tissue inflammatory response, nerve injury and death were significantly inhibited, while the factors related to tissue regeneration were activated by BSiX. (FIG. 14).

The associated cytokine receptors also responded similarly (FIG. 15).

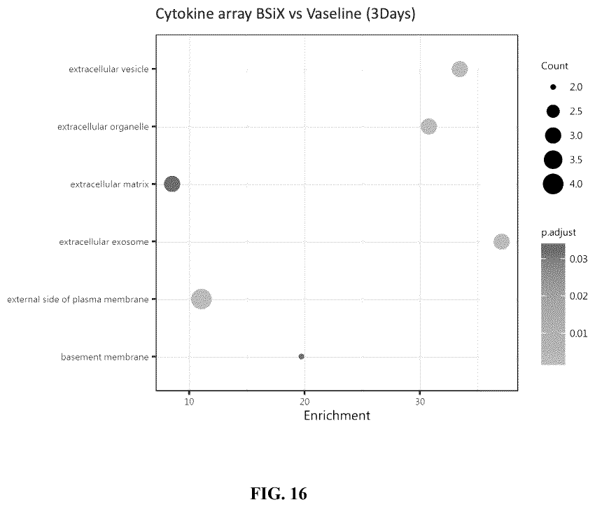

Notably, some factors associated with the extracellular matrix were activated, while ECM degradation was inhibited (FIG. 16).

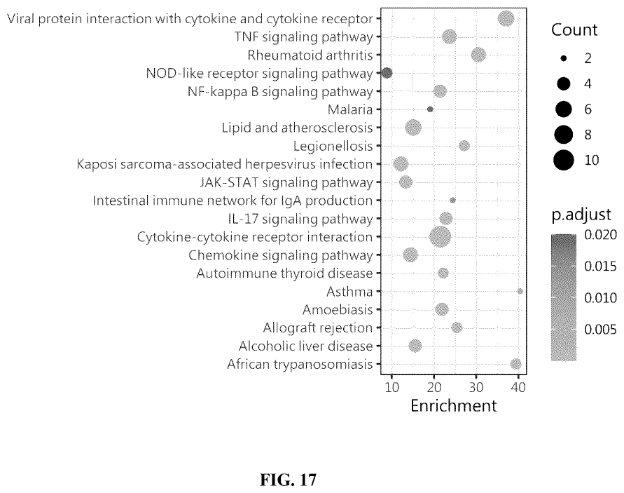

Furthermore, many cellular signaling pathways were regulated, especially the activity of cytokine receptors that interacted with viral proteins was increased (FIG. 17). This result suggests that BSiX has potential antiviral ability.

### Twenty-Day Comparison Results Between BSiX and Vaseline Gauze Groups

Twenty days post the burn injury, the results showed that a total of 7 cytokines were increased by BSiX, including INF-r, Leptin, CD86, IL1, IL-1b, PDGF-AA, Fas-L. The increase of PDGF-AA was especially obvious, significantly higher than the 3-day increase. This has important biological implications, indicating that BSiX can promote tissue regeneration, and the effect persists.

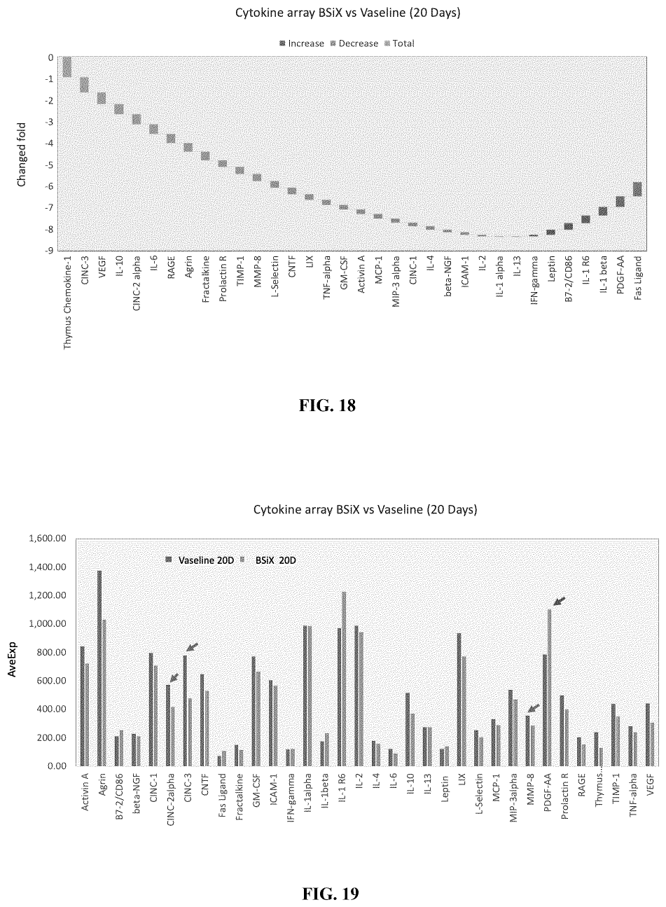

In addition, BSiX significantly inhibited 27 endogenous cytokines, the most important of which were CINC-2, MMP8, and CINC3, which were all related to tissue inflammation, indicating that BSiX can significantly inhibit tissue inflammation and persist (FIG. 18 & FIG. 19).

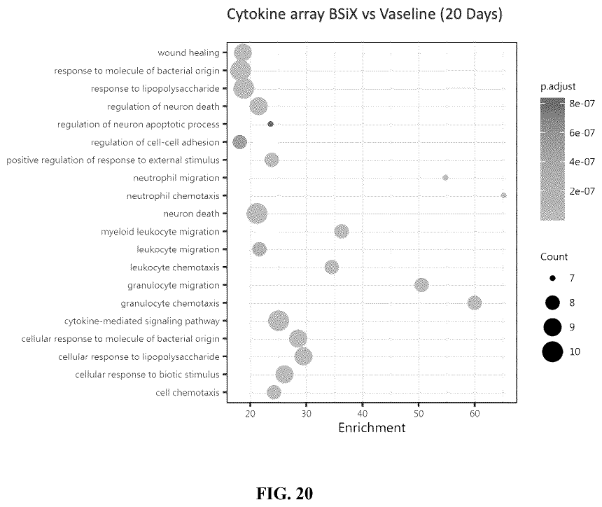

Also, through the in-depth analysis of these endogenous cytokines, it was found that the factors associated with tissue damage, bacterial infection, and tissue inflammatory responses, leukocyte infiltration were significantly inhibited, while leukocyte chemotactic factors were activated. Of particular note is the inhibition of intercellular adhesion signaling molecules, which are important for tissue regeneration and shaping (FIG. 20).

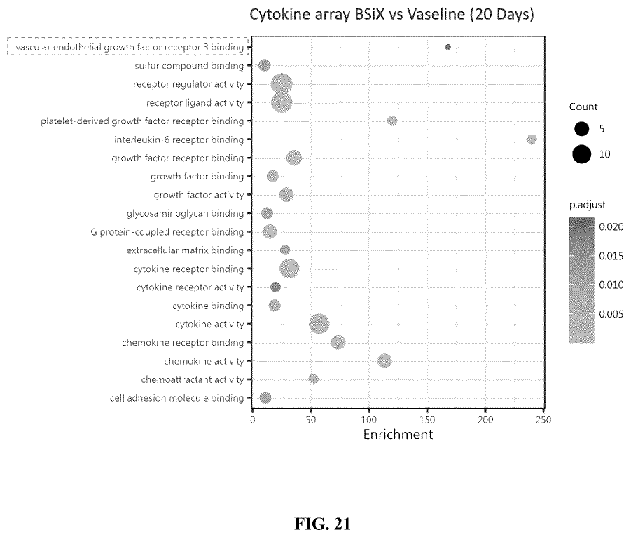

The associated cytokine receptors also responded similarly, notably the activation of VEGF receptor 3 by BSIX, suggesting that BSiX can promote angiogenesis (FIG. 21).

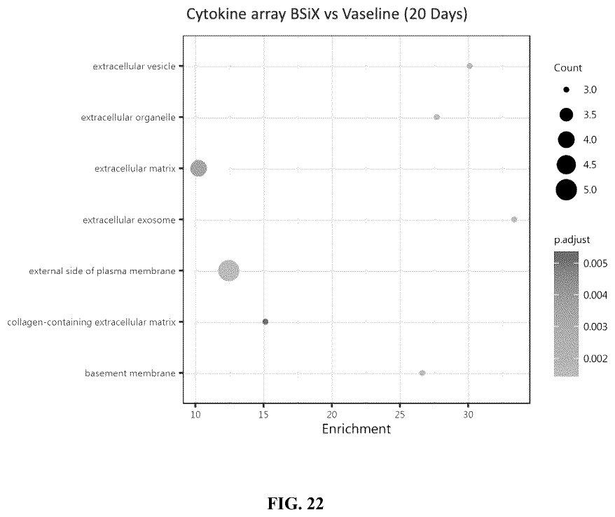

Notably, some factors associated with extracellular matrix were activated at 20 days, while ECM degradation was inhibited, and collagen-related extracellular matrix activity was reduced, suggesting that BSIX regulates tissue shaping (FIG. 22).

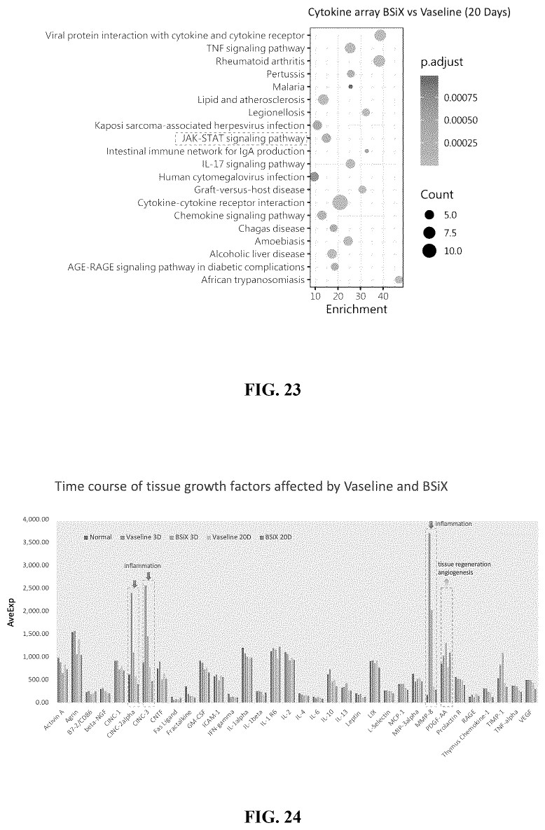

Moreover, many signaling pathways were regulated and similar to 3 days, especially the activity of cytokine receptors interacting with viral proteins was enhanced and persisted (FIG. 23). This result suggests that BSiX has potential antiviral ability.

At the same time, BSiX can significantly inhibit the factors related to tissue inflammation, among which the important ones are CINC-2, MMP8, CINC3, and BSiX inhibits the inflammatory response of the tissue is also persistent, and the effect is stronger in the early stage, 3 days is stronger than 20 days (FIG. 24).

Presented as gel, the BloodSTOP iX Wound Heal serves as a scaffold, giving the patient's native cells a place to live, thrive and rebuild tissue. Without cross-linking, the open scaffold allows for cellular infiltration and capillary growth. At the same time, VEGF and PDGF, the cytokines stimulated by BloodSTOP iX Wound Heal, were released from the host cells and rebuilt well-vascularized new skin tissues.

The BloodSTOP iX Wound Heal modulated endogenous cytokines, where the BloodSTOP gel provide perfect microenvironment for BloodSTOP iX Wound Heal molecules to bind to cellular receptors on cell membranes to activate cellular signaling for modulating endogenous cytokines, such as VEGF, PDGF, EGF, FGF2, FGF7, TGF-β1 and TGF-β3 etc. VEGF and PDGF, the cytokines stimulated by B BloodSTOP iX Wound Heal, were released from the host cells and rebuilds well-vascularized new tissues. At the same time, FGF7 and others, the cytokine stimulated by BloodSTOP iX Wound Heal, protected tissue from injury and decrease apoptosis. More importantly, TGFβ3 and others, the cytokine stimulated by BloodSTOP iX Wound Heal, facilitated cellular transformation and infiltration to enhance skin regeneration and re-epithelialization.

BSiX can significantly increase the growth factors related to tissue regeneration and repair such as PDGF-AA, thereby promoting tissue regeneration, and this effect persists over time and is higher at 20 days than at 3 days. The BSiX modulated endogenous cytokines, where the BloodSTOP iX Wound Heal gel provide perfect microenvironment for BloodSTOP iX Wound Heal molecules to bind to cellular receptors on cell membranes to activate cellular signaling for modulating endogenous cytokines, such PDGF-AA, etc. At the same time, BloodSTOP iX Wound Heal decrease inflammatory cytokines, including MMP8, CINC1 and CINC3, to reduce the infection and inflammation risk.

As used herein, the terms “comprises,” “comprising”, or “having” when used to define compositions and methods, are intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Applicant's disclosure is described herein in preferred embodiments with reference to the figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A composition for use in modulating one or more growth factors for skin and/or tissue regeneration, comprising a biocompatible carboxymethyl cellulose (CMC).

2. The composition of claim 1, wherein the skin or tissue comprises an injury or burn.

3. The composition of claim 2, wherein tissue regeneration is promoted by dermal and/or stem cell proliferation.

4. The composition of claim 1, wherein the biocompatible carboxymethyl cellulose (CMC) is characterized by a degree of fabric substitution in the range about 0.1 to about 0.99.

5. The composition of claim 1, wherein the biocompatible carboxymethyl cellulose (CMC) is characterized by a degree of polymerization is from about 50 to about 2,000.

6. The composition of claim 5, wherein the biocompatible carboxymethyl cellulose (CMC) is characterized by a sodium content in the range from about 6.5% to about 9.5%.

7. The composition of claim 1, for use in stimulating endogenous cytokines VEGF and/or PDGF.

8. The composition of claim 1, for use in inhibiting one or more inflammatory cytokines.

9. A medical device comprising the composition according to claim 1.

10. The medical device of claim 9, further comprising one or more bioactive agent(s) that stimulates wound healing.

11. The medical device of claim 10, further comprising one or more bioactive agent(s) that prevents or reduces infection.

12. The medical device of claim 10, further comprising one or more anti-fibrinolysis agents.

13. The medical device of claim 10, further comprising one or more adhesive agents.

14. The medical device of claim 10, further comprising one or more coagulation factors.

15. The medical device of claim 9, in a form selected from powders, fibers, webs, nonwoven cloths, sponges, films, capsules, pellets, columns, plugs and colloids.

16. A kit for skin or tissue injury or burn treatment, comprising a medical device of claim 1.

17. A method for modulating one or more growth factors for skin and/or tissue regeneration, comprising applying a composition or a medical device of claim 1 to a patient in need thereof at an injury or burn site.

18. A method for modulating one or more growth factors for promoting dermal and/or stem cell proliferation, comprising applying a composition or a medical device of claim 1 to a patient in need thereof at an injury or burn site.

19. The method of claim 17, wherein the composition or device is applied on the patient for about 1 day to about 28 days.

20. The method of claim 18, wherein the composition or device is applied on the patient for about 1 day to about 28 days.
